(19) United States
(12) Patent Application Publication
(10) Pub. No.: US 2025/0269099 A1
(43) Pub. Date: Aug. 28, 2025

(54) Method for Operating Blood Treatment System and Corresponding Blood Treatment System

(71) Applicant: Fresenius Medical Care Deutschland GmbH, Bad Homburg (DE)

(72) Inventors: Kee Jor, Bad Homburg (DE); Jiaming Dong, Bad Homburg (DE); Yicheng Peng, Bad Homburg (DE)

(21) Appl. No.: 18/858,138

(22) PCT Filed: Mar. 1, 2023
(86) PCT No.: PCT/CN2023/079006

§ 371 Date: Oct. 18, 2024

(30) Foreign Application Priority Data
Apr. 21, 2022 (WO) PCT/CN2022/088100

(51) Int. Cl. A61M 1/16; A61M 1/34; A61M 39/22; G16H 40/63

(52) U.S. Cl. CPC A61M 1/1603; A61M 1/1625; A61M 1/1635; A61M 1/3403; A61M 1/3424; A61M 1/3441; A61M 39/22; G16H 40/63; A61M 2202/0021; A61M 2202/0413; A61M 2205/10; A61M 2205/3327; A61M 2205/3334; A61M 2205/3365; A61M 2205/50; A61M 2230/005; A61M 2230/207

(57) ABSTRACT

Herein disclosed is a method for operating a blood treatment system comprising a dialyzer, wherein the dialyzer comprises a first cavity region, in which a first blood chamber is defined at least partially by a first semi-permeable membrane, and a second cavity region, in which a second blood chamber is defined at least partially by a second semi-permeable membrane, the first blood chamber and the second blood chamber are communicated with each other, and the method at least comprises: adjusting a flow characteristic of a flow path between the first cavity region and the second cavity region at least by controlling an on-off operation of an on-off valve disposed on the flow path, in order to control a substitution rate of a medical fluid externally supplied into the second cavity region. Also disclosed is a corresponding blood treatment system.

### CROSS-REFERENCE TO RELATED APPLICATIONS

The present application is the national stage entry of International Patent Application No. PCT/CN2023/079006, filed on Mar. 1, 2023, the disclosure of which is incorporated herein by reference.

### TECHNICAL FIELD

The disclosure relates to a method for operating a blood treatment system comprising a dialyzer and to a corresponding blood treatment system.

### BACKGROUND

A dialysis treatment is a procedure for removing toxic substances and metabolites normally removed by the kidneys, and for aiding in regulation of fluid and electrolyte balance.

The dialysis treatment may be carried out by various types of dialysis procedures, such as a hemodialysis (HD) and a peritoneal dialysis (PD). The hemodialysis is usually executed by using a hemodialysis machine.

Standard hemodialysis (HD) generally uses diffusion to remove waste products from a patient's blood. A diffusive gradient that occurs within the semi-permeable dialyzer between the blood and dialysate may cause diffusion. Further, hemofiltration (HF) is an alternative renal replacement therapy that relies on a convective transport of toxins from the patient's blood. This therapy is accomplished by directly or indirectly adding substitution or replacement fluid to the extracorporeal circuit during treatment. That substitution fluid and the fluid accumulated by the patient between dialysis sessions is ultrafiltered over the course of the HF treatment, thereby providing a convective transport mechanism that is particularly beneficial in removing middle and large molecules.

Hemodiafiltration (HDF) is a treatment mode that combines convective and diffusive clearances and thus may represent a gold standard in the field of replacement of renal function by dialysis. HDF uses dialysate to flow through a dialyzer, for example a high flux dialyzer, similar to standard hemodialysis, providing diffusive clearance. In addition, the substitution solution, which also may be dialysate, is provided directly or indirectly to the extracorporeal circuit, providing convective clearance.

### SUMMARY

In one aspect, a dialyzer has a simplified structure and reduced cost. Such a dialyzer may be especially useful for HDF. Moreover, for HDF, a corresponding controlling method may provide simplified operation and reduced hardware requirements.

One aspect of the disclosure is to provide an improved method for operating a blood treatment system comprising a dialyzer and a corresponding blood treatment system.

According to a first aspect, provided is a method for operating a blood treatment system comprising a dialyzer, wherein the dialyzer comprises a first cavity region, in which a first blood chamber is defined at least partially by a first semi-permeable membrane, and a second cavity region, in which a second blood chamber is defined at least partially by a second semi-permeable membrane, the first blood chamber and the second blood chamber are communicated with each other, and the method at least comprises: adjusting a flow characteristic of a flow path between the first cavity region and the second cavity region at least by controlling an on-off operation of an on-off valve disposed on the flow path, in order to control a substitution rate of a medical fluid externally supplied into the second cavity region.

According to an optional embodiment of the present disclosure, the flow characteristic is adjusted by controlling an on/off time ratio of the on-off valve.

According to an optional embodiment of the present disclosure, the on-off valve is configured as a solenoid valve.

According to an optional embodiment of the present disclosure, the dialyzer comprises: a housing defining a cavity comprising the first cavity region and the second cavity region, which are communicated with each other by a pass-through passage; a plurality of hollow fiber membranes extending from the first cavity region to the second cavity region across the passage so as to form the first semi-permeable membrane and the second semi-permeable membrane; a flow restricting structure located in an area of the passage so as to restrict flow of the medical fluid between the first cavity region and the second cavity region; a first port and a second port each fluidly communicated with the first cavity region; and a third port and a fourth port each fluidly communicated with the second cavity region; the housing has a substantially cylindrical configuration and the first port, the second port, the third port and the fourth port are disposed on the housing sequentially in an axially spaced manner from each other in an axial direction, in some instances in a row; and the second port and the third port are fluidly connected at least by the on-off valve.

According to an optional embodiment of the present disclosure, the method further comprises: controlling momentary peak pressure acting on the hollow fiber membranes within the second cavity region via a flow restriction connected fluidly in parallel with the on-off valve.

According to an optional embodiment of the present disclosure, the flow restriction is configured as a variable flow restriction.

According to an optional embodiment of the present disclosure, the method further comprises: adjusting the substitution rate by further controlling the flow restriction.

According to an optional embodiment of the present disclosure, the method further comprises: adjusting a supplying speed of the medical fluid into the second cavity region at least based on the on-off operation of the on-off valve.

According to an optional embodiment of the present disclosure, the supplying speed is reduced when the on-off valve is closed.

According to an optional embodiment of the present disclosure, the blood treatment system further comprises a fluid supply device, eg. a balancing chamber device for supplying the medical fluid towards the dialyzer and the supplying speed is controlled by adjusting a speed of a flow pump of the fluid supply device.

According to an optional embodiment of the present disclosure, wherein the method further comprises: indirectly determining a first transmembrane pressure of the first cavity region based on a first pressure at the first port, a second pressure at the second port, a third pressure at the third port, a fourth pressure at the fourth port and a first set of additional parameters at least including a supplying flow rate of the medical fluid, the substitution rate and a blood flow rate, according to a first predetermined functional relationship; and/or indirectly determining a second transmembrane pressure of the second cavity region based on a first pressure at the first port, a second pressure at the second port, a third pressure at the third port, a fourth pressure at the fourth port and a second set of additional parameters at least including a supplying flow rate of the medical fluid, the substitution rate and a blood flow rate, according to a second predetermined functional relationship.

According to an optional embodiment of the present disclosure, the first set of additional parameters further comprise at least one of an ultrafiltration factor and a hematocrit level; and/or the second set of additional parameters further comprise at least one of an ultrafiltration factor and a hematocrit level.

According to an optional embodiment of the present disclosure, the first predetermined functional relationship is established by experiments and/or simulations; and/or the second predetermined functional relationship is established by experiments and/or simulations.

According to an optional embodiment of the present disclosure, the method further comprises: monitoring the substitution rate by a flow sensor for detecting a flow rate of the medical fluid flowing from the second cavity region towards the first cavity region.

According to a another aspect, provided is a blood treatment system, configured to be adapted to perform hemodialysis, hemofiltration and hemodiafiltration, comprising: a memory with computer-readable program instructions stored thereon; and a processor for executing the computer-readable program instructions so as to perform the method according to any one of the above embodiments.

According to some exemplary embodiments of the present disclosure, the blood treatment system can be operated or controlled more easily, and the blood treatment system has a simple arrangement.

### BRIEF DESCRIPTION OF THE DRAWINGS

The disclosure and advantages thereof will be further understood by reading the following detailed description of some preferred exemplary embodiments with reference to the drawings, in which:

FIG. 1 schematically shows a sectional view of a dialyzer according to an exemplary embodiment of the present disclosure.

FIG. 2 schematically shows a sectional view of the dialyzer according to another exemplary embodiment of the present disclosure.

FIG. 3 shows a blood treatment system using the dialyzer shown in FIG. 2 and a balancing chamber device for supplying medical fluid towards the dialyzer.

FIG. 4 shows a graph illustrating a relationship of (P3-P5)/(P3-P4) versus Qsub/Qb tested for a certain dialyzer at three different blood flow rates of 350 ml/min, 400 ml/min and 450 ml/min. FIG. 5 shows a graph illustrating a relationship of (P1-P6)/(P1-P2) versus Qsub/Qm tested for a certain dialyzer at three different blood flow rates of 350 ml/min, 400 ml/min and 450 ml/min.

### DETAILED DESCRIPTION

Some exemplary embodiments of the present disclosure will be described hereinafter in more details with reference to the drawings to better understand the basic concept and advantages of the present disclosure.

According to an aspect of the disclosure, herein firstly proposed is a dialyzer for blood treatment, comprising: a housing defining a cavity comprising a first cavity region and a second cavity region which are communicated with each other by a pass-through passage; a plurality of hollow fiber membranes extending from the first cavity region to the second cavity region across the passage; a flow restricting structure located in an area of the passage so as to restrict flow of medical fluid, for example dialysate, between the first cavity region and the second cavity region; a first port and a second port fluidly communicated with the first cavity region; and a third port and a fourth port fluidly communicated with the second cavity region.

Here, both the first cavity region and the second cavity region are formed within a common housing.

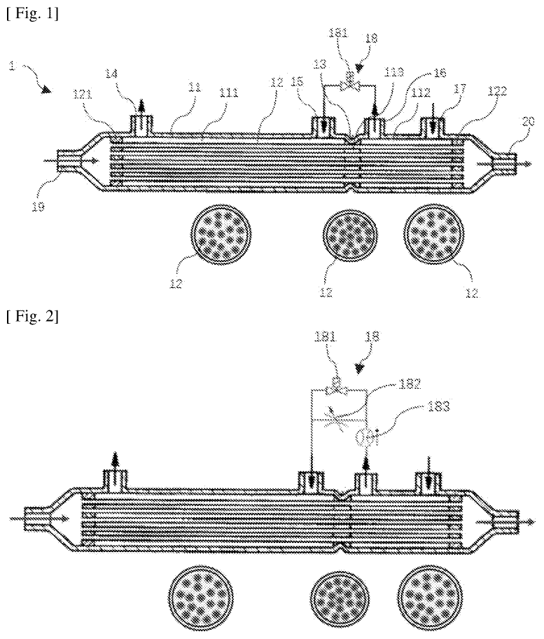

FIG. 1 schematically shows a sectional view of the dialyzer 1 according to an exemplary embodiment of the present disclosure.

As shown in FIG. 1, the dialyzer 1 may comprise: a substantially cylindrical housing 11 defining a cavity comprising a first cavity region 111 and a second cavity region 112 which may be communicated with each other by a pass-through passage 113, wherein the first cavity region 111, the second cavity region 112 and the pass-through passage 113 may be arranged coaxially; a plurality of hollow fiber membranes 12 extending from the first cavity region 111 to the second cavity region 112 across the passage 113; a flow restricting structure 13 formed in an area of the passage 113 so as to restrict flow of the medical fluid from the second cavity region 112 to the first cavity region 111; a first port 14, as a dialysate (effluent) outlet port, also called as an ultrafiltrate port in some cases, and a second port 15, as a dialysate inlet port, fluidly communicated with the first cavity region 111; and a third port 16, as a further dialysate outlet port, and a fourth port 17, as a further dialysate inlet port, fluidly communicated with the second cavity region 112. In this case, the dialyzer 1 comprises two sub-dialyzers using the common housing 11.

The dialyzer 1 may have a blood inlet port 19 and a blood outlet port 20. During the blood treatment, the blood to be treated flows into the blood inlet port 19 via an arterial blood line (not shown) from a patient, flow through lumens of the hollow fiber membranes 12, and then the treated blood flows out of the blood outlet port 20 via a venous blood line (not shown) back to the patient, while the dialysate flows in the first cavity region 111 and the second cavity region 112. Because the fact that the basic working principle of the dialyzer 1 itself during the blood treatment is known, the description will mainly focus on some parts associated with the present disclosure to achieve a corresponding simplification.

In FIG. 1, as shown in corresponding arrows, the dialysate flows within the housing 11 in a countercurrent manner, which can achieve better blood treatment, particularly more intense diffusion across the hollow fiber membranes 12. However, the skilled person in the art may understand that this is just an example.

As shown in FIG. 1, the hollow fiber membranes 12 are potted only at their opposite ends 121 and 122. That means that each of the hollow fiber membranes 12 is continuous across the passage 113 without any potting area in the passage 113.

According to an exemplary embodiment of the present disclosure, as shown in FIG. 1, the passage 113 may be configured as a narrowed region defined at least partially by the flow restricting structure 13. In this narrowed region, the hollow fiber membranes 12 are denser due to radial compression on them.

FIG. 1 also shows cross-sectional views of the dialyzer 1 at three corresponding different areas in its lower portion. As can be seen from FIG. 1, the hollow fiber membranes 12 are closer to each other in the passage 113, which means that the dialysate is more difficult to, and even cannot, flow through gaps between any adjacent two of the hollow fiber membranes 12.

According to an exemplary embodiment of the present disclosure, as shown in FIG. 1, the flow restricting structure 13 may be configured to be integrally formed on the housing 11. As can be seen from FIG. 1, the flow restricting structure 13 is formed as a portion of the housing 11. In this case, the flow restricting structure 13 may be formed during injection molding of the housing 11 (which is often made of plastic). As shown in FIG. 1, the flow restricting structure 13 may comprise a protrusion formed during injection molding of the housing 11. The protrusion, in some instances, is formed by a curved wall portion of the housing 11. Such a configuration may be formed easily during injection molding of the housing 11.

The skilled person in the art may understand that the flow restricting structure 13 also may be configured to be disposed within the housing 11. Particularly, the flow restricting structure 13 may be configured as a separate part and then be positioned relative to, for example be attached to the housing 11 in any suitable manner. For example, the hollow fiber membranes 12 may be loaded within the flow restricting structure 13 and then they together be loaded into the housing 11.

According to an exemplary embodiment of the present disclosure, at least one, of the first port 14, the second port 15, the third port 16 and the fourth port 17 may be molded integrally with the housing 11, which is very advantageous.

However, if the flow restricting structure 13 is pre-molded as shown in FIG. 1, it may be difficult to load the hollow fiber membranes 12 through the passage 113 due to a limited size of the passage 113. Thus, it may be advantageous that the flow restricting structure 13 is formed finally at least after the hollow fiber membranes 12 are loaded into the cavity of the housing 11.

Thus, according to an exemplary embodiment of the present disclosure, the flow restricting structure 13 may be formed at least partially by a shrinkable structure which can be shrunk after injection molding of the housing 11, particularly after loading of the hollow fiber membranes 12.

In some instances, the shrinkable structure may be a heat-shrinkable structure, particularly a heat-shrinkable tube. Shrinking of the shrinkable structure can be preformed easily for the heat-shrinkable structure. Of course, the present disclosure is not limited hereto. For example, the shrinkable structure can be shrunk by lights having a specific wavelength.

According to an exemplary embodiment of the present disclosure, the heat-shrinkable tube may form at least a portion, for example an axial segment of the housing 11 in the area of the passage 113. That is, at least this segment may be formed by a heat-shrinkable material.

The skilled person in the art may understand that the heat-shrinkable tube may be firstly disposed within the housing 11 and then be shrunk, particularly after loading of the hollow fiber membranes 12.

Generally, the hollow fiber membranes 12 will expand when exposed to water, which means that on the one hand the flow restricting structure 13 should be designed while considering expansion property of the hollow fiber membranes 12 in use to avoid excessive compression of the hollow fiber membranes 12 and thus damage to the hollow fiber membranes 12 or adversely affecting flowing of the blood through the lumens of the hollow fiber membranes 12, but on the other hand, we can take advantage of this to help to restrict flow of the medical fluid from the second cavity region 112 to the first cavity region 111, particularly considering that a priming process should be performed before starting the actual blood treatment.

Thus, it may be sufficient to configure the flow restricting structure 13 to tightly hold the hollow fiber membranes 12 relatively as shown in FIG. 1.

According to an exemplary embodiment of the present disclosure, the first port 14 or the second port 15 may be connected with the third port 16 or the fourth port 17 through a fluid flow control device 18 to control flow of the medical fluid therebetween. FIG. 1 just shows an example in which the second port 15 and the third port 16 are connected by the fluid flow control device 18.

The dialyzer 1 has the first cavity region 111 and the second cavity region 112, between which the flow restricting structure 13 is located, such that pressure profiles of the dialysate within the first cavity region 111 and the second cavity region 112 can be controlled or adjusted in a more flexible manner to achieve a desired treatment goal, which may vary with different treatment conditions, particularly for different patients. Thus, the present disclosure does not intend to expel any other possibilities, for example connecting of the third port 16 with the first port 14, although the specific connection manner as shown in FIG. 1 may be advantageous for most of blood treatment modes.

As shown in FIG. 1, the first port 14, the second port 15, the third port 16 and the fourth port 17 may be disposed on the housing 11 sequentially in an axially spaced manner from each other in an axial direction. In some instances, each port is positioned in a row. In some instances, at least one, of the first port 14, the second port 15, the third port 16 and the fourth port 17 may extend laterally, as shown in FIG. 1.

As can be seen from FIG. 1, the second port 15 and the third port 16 may be disposed at opposite side of the flow restricting structure 13 respectively and are adjacent to each other.

According to an exemplary embodiment of the present disclosure, as shown in FIG. 1, the fluid flow control device 18 may comprise an on-off valve 181, which can switch on or off flowing of the dialysate from the third port 16 toward the second port 15.

According to an exemplary embodiment of the present disclosure, the on-off valve 181 may be a solenoid valve, which can be controlled easily as desired. In this case, the solenoid valve can be controlled flexibly in on/off cycles.

However, such a control manner may cause undesirable momentary stress across the hollow fiber membranes 12. In this case, it may be advantageous to provide a flow restriction 182 connected in parallel with the on-off valve 181, which is exemplarily shown in FIG. 2, which is the same as FIG. 1 except for the different fluid flow control device 18. The flow restriction 182 can partially divert some dialysate and thus be effective to lower momentary peak pressure acting on the hollow fiber membranes 12. Use of the flow restriction 182 also can optimize the HDF treatment process.

The skilled person in the art may understand that the flow restriction 182 also may be used instead of the on-off valve 181. Only use of the flow restriction 182 also can achieve the HDF treatment mode.

According to an exemplary embodiment of the present disclosure, the flow restriction 182 may be a variable flow restriction. In some instances, the variable flow restriction can be controlled automatically.

As shown in FIG. 2, according to an exemplary embodiment of the present disclosure, the fluid flow control device 18 may further comprise a flow sensor 183 for detecting a flowrate of the dialysate from the third port 16 to the second port 15. In some instances, the flow sensor 183 may be disposed upstream of the on-off valve 181 and the flow restriction 182.

According to an exemplary embodiment of the present disclosure, the fluid flow control device 18 may be integrated as a shunt interlock assembly, which will facilitate assembling of the fluid flow control device 18 onto the second port 15 and the third port 16.

A controller (not shown) may be provided to control the fluid flow control device 18 to adapt to various blood treatments.

According to another aspect of the present disclosure, further proposed is a system for blood treatment, i.e., a blood treatment system, comprising the dialyzer 1 described above, a blood line (not shown) and a medical fluid line (not shown), wherein the blood line and medical fluid line are fluidly connected with the dialyzer 1.

According to an exemplary embodiment of the present disclosure, the system may be configured to perform hemodialysis, hemofiltration, hemodiafiltration and any combination thereof.

The above mainly describes the structure or arrangement of the dialyzer, and the following will describe in details how to operate the blood treatment system especially comprising the above dialyzer. However, the skilled person in the art can understand that such an operation is also suitable for other similar dialyzer. Thus, in terms of operation, the following description does not only rely on the above kind of dialyzer. Of course, such an operation may be especially suitable for the kind of dialyzer.

According to a further aspect of the present disclosure, provided is a method for operating a blood treatment system comprising the dialyzer 1 (which just may be the above dialyzer, not must be the above dialyzer), wherein the dialyzer 1 comprises the first cavity region 111, in which a first blood chamber is defined at least partially by a first semi-permeable membrane, and the second cavity region 112, in which a second blood chamber is defined at least partially by a second semi-permeable membrane, the first blood chamber and the second blood chamber are communicated with each other, and the method at least comprises: adjusting a flow characteristic of a flow path between the first cavity region 111 and the second cavity region 112 at least by controlling an on-off operation of the on-off valve 181 disposed on the flow path, in order to control a substitution rate Qsub of the medical fluid externally supplied into the second cavity region 112. Specifically, the flow characteristic from the second cavity region 112 towards the first cavity region 111 may be adjusted as desired by controlling the on-off operation of the on-off valve 181. Such an adjustment can be achieved easily by opening and closing the on-off valve 181.

The substitution rate Qsub of the medical fluid within the second cavity region 112 may be expressed as a supplying flow rate Qm of the medical fluid into second cavity region 112 for example through the fourth port 17 minus a flow rate Qf of the medical fluid flowing out of the second cavity region 112, which is related closely with an operation status of the on-off valve 181. That is to say Qsub=Qm-Qf. Thus, it is possible to control the substitution rate Qsub of the medical fluid by controlling the on-off operation of the on-off valve 181.

In some instances, the flow characteristic is adjusted by controlling an on/off time ratio of the on-off valve 181. The higher the on/off time ratio, the lower a pressure difference between the medical fluid and the blood and thus the lower an amount of the medical fluid entering the blood across the semi-permeable membrane. Roughly speaking, the substitution rate Qsub is substantially in negative proportion to the on/off time ratio.

As shown in FIG. 1, the plurality of hollow fiber membranes 12 continuously extending from the first cavity region 111 to the second cavity region 112 across the passage 113 can form the first semi-permeable membrane and the second semi-permeable membrane.

As mentioned above, the flow restriction 182 can partially divert some dialysate and thus be effective to lower momentary peak pressure acting on the hollow fiber membranes 12. Thus, according to an exemplary embodiment of the present disclosure, the method further comprises: controlling the momentary peak pressure acting on the hollow fiber membranes 12 within the second cavity region 112 via the flow restriction 182.

When the flow restriction 182 is adjustable, it may be desirable to adjust the substitution rate Qsub by further controlling the flow restriction 182, in addition to the on-off valve 181.

According to an exemplary embodiment of the present disclosure, the method further comprises: adjusting a supplying speed of the medical fluid into the second cavity region 112 at least based on the on-off operation of the on-off valve 181. If the medical fluid is supplied too fast towards the fourth port 17, it will result in unfavorable high stress on the semi-permeable membrane especially in a hemofiltration mode when the on-off valve 181 is closed. In this case, the supplying speed is reduced when the on-off valve 181 is closed. In some instances, a pressure of the medical fluid remains unchanged.

It can be understood that it is more advantageous to adjust of the supplying speed of the medical fluid in combination with provision and adjusting of the flow restriction 182.

The above have mentioned that the flow sensor 183 can detect the flow rate of the dialysate from the third port 16 to the second port 15, i.e., a flow rate of the medical fluid flowing from the second cavity region 112 towards the first cavity region 111. Thus, it can be understood that the substitution rate Qsub can be monitored by the flow sensor 183.

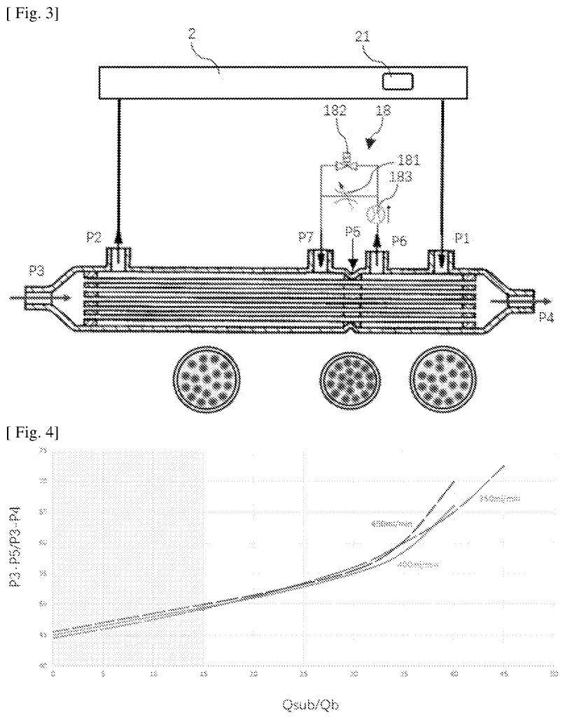

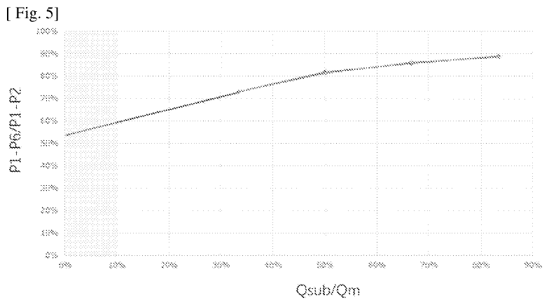

FIG. 3 shows the blood treatment system using the dialyzer 1 shown in FIG. 2 and a balancing chamber device 2 for supplying the medical fluid towards the dialyzer 1, wherein the balancing chamber device 2 is shown schematically. In this case, the supplying speed can be adjusted by adjusting a speed of a flow pump 21 of the balancing chamber device 2.

Below, we will further describe how to monitor a transmembrane pressure, which is very important to control the treatment process.

As shown in FIG. 3, some marks are provided to indicate pressures of the medical fluid or the blood at corresponding areas. According to definition of the transmembrane pressure, for the first cavity region 111, the first transmembrane pressure TMP1 can be calculated according to the equation (1):

$$TMP 1 = ( P 3 + P 5 ) / 2 - ( P 2 + P 7 ) / 2 \qquad ( 1 )$$

For the second cavity region 112, the second transmembrane pressure TMP2 can be calculated according to the equation (2):

$$TMP 1 = ( P 5 + P 4 ) / 2 - ( P 1 + P 6 ) / 2 \qquad ( 2 )$$

Thus, in principle, seven pressure sensors are required to measure these pressures and then directly determine the first transmembrane pressure TMP1 and the second transmembrane pressure TMP2. Too many sensors and pressure lines equipped for them are tedious for the blood treatment. Particularly for the dialyzer 1 shown in FIG. 3, the hollow fiber membranes 12 is continuous and thus it is difficult to directly measure the pressure P5.

According to the present disclosure, the first transmembrane pressure TMP1 and the second transmembrane pressure TMP2 are indirectly determined.

For most hemodialysis machines, they are usually equipped with corresponding pressure sensors which can measure P1, P2, P3 and P4. Thus, a challenge is how to determine the first transmembrane pressure TMP1 and the second transmembrane pressure TMP2 only using P1, P2, P3 and P4 and other some available parameters to reduce hardware requirements.

For the specific dialyzer with known properties, it can be understood that the supplying flow rate Qm of the medical fluid, a blood flow rate Qb, and the substitution rate Qsub of the medical fluid will affect the pressure characteristics of the medical fluid and the blood in the first cavity region 111 and the second cavity region 112. Thus, it is desired to further introduce these parameters when determining the first transmembrane pressure TMP1 and the second transmembrane pressure TMP2 only with P1, P2, P3 and P4.

FIG. 4 shows a graph illustrating a relationship of (P3-P5)/(P3-P4) versus Qsub/Qb tested for a certain dialyzer at three different blood flow rates of 350 ml/min, 400 ml/min and 450 ml/min. It can be seen that P5 can be calculated from P3, P4, Qsub and Qb.

FIG. 5 shows a graph illustrating a relationship of (P1-P6)/(P1-P2) versus Qsub/Qm tested for a certain dialyzer at three different blood flow rates of 350 ml/min, 400 ml/min and 450 ml/min. It is found that the blood flow rate Qb is substantially irrelevant to (P1-P6)/(P1-P2). It can be seen that P6 can be calculated from P1, P2, Qsub and Qm.

It can be understood by the skilled person in the art that P7 also can be determined indirectly for a certain dialyzer by experiments and/or simulations.

Once P5, P6 and P7 are calculated, TMP1 and TMP2 can be directly determined by the equations (1) and (2).

In addition, an ultrafiltration factor and a hematocrit level of the blood to be treated also will affect these pressures and thus can be considered when determining the first transmembrane pressure TMP1 and the second transmembrane pressure TMP2.

Especially for a specific dialyzer, functional relationships between these parameters can be predetermined by various suitable experiments and/or simulations. The present disclosure does not intend to limit thereto, as long as not all of PI-P7 are necessary and used. These functional relationships are stored in a query table.

While certain embodiments have been described, these embodiments have been presented by way of example only, and are not intended to limit the scope of the disclosure. The attached claims and their equivalents are intended to cover all the modifications, substitutions and changes as would fall within the scope and spirit of the disclosure.

1-15. (canceled)

16. A method for operating a blood treatment system comprising a dialyzer, wherein the dialyzer comprises a first cavity region, in which a first blood chamber is defined at least partially by a first semi-permeable membrane, and a second cavity region, in which a second blood chamber is defined at least partially by a second semi-permeable membrane, the first blood chamber and the second blood chamber are communicated with each other, and the method at least comprises:
adjusting a flow characteristic of a flow path between the first cavity region and the second cavity region at least by controlling an on-off operation of an on-off valve disposed on the flow path, in order to control a substitution rate of a medical fluid externally supplied into the second cavity region.

17. The method according to claim 16, wherein
the flow characteristic is adjusted by controlling an on/off time ratio of the on-off valve.

18. The method according to claim 16, wherein
the on-off valve is configured as a solenoid valve.

19. The method according to claim 16, wherein
the dialyzer comprises: a housing defining a cavity comprising the first cavity region and the second cavity region, which are communicated with each other by a pass-through passage; a plurality of hollow fiber membranes extending from the first cavity region to the second cavity region across the passage so as to form the first semi-permeable membrane and the second semi-permeable membrane; a flow restricting structure located in an area of the passage so as to restrict flow of the medical fluid between the first cavity region and the second cavity region; a first port and a second port each fluidly communicated with the first cavity region; and a third port and a fourth port each fluidly communicated with the second cavity region;
the housing has a substantially cylindrical configuration and the first port, the second port, the third port and the fourth port are disposed on the housing sequentially in an axially spaced manner from each other in an axial direction, preferably in a row; and
the second port and the third port are fluidly connected at least by the on-off valve.

20. The method according to claim 17, wherein
the dialyzer comprises: a housing defining a cavity comprising the first cavity region and the second cavity region, which are communicated with each other by a pass-through passage; a plurality of hollow fiber membranes extending from the first cavity region to the second cavity region across the passage so as to form the first semi-permeable membrane and the second semi-permeable membrane; a flow restricting structure located in an area of the passage so as to restrict flow of the medical fluid between the first cavity region and the second cavity region; a first port and a second port each fluidly communicated with the first cavity region, and a third port and a fourth port each fluidly communicated with the second cavity region;
the housing has a substantially cylindrical configuration and the first port, the second port, the third port and the fourth port are disposed on the housing sequentially in an axially spaced manner from each other in an axial direction, preferably in a row; and
the second port and the third port are fluidly connected at least by the on-off valve.

21. The method according to claim 18, wherein
the dialyzer comprises: a housing defining a cavity comprising the first cavity region and the second cavity region, which are communicated with each other by a pass-through passage; a plurality of hollow fiber membranes extending from the first cavity region to the second cavity region across the passage so as to form the first semi-permeable membrane and the second semi-permeable membrane; a flow restricting structure located in an area of the passage so as to restrict flow of the medical fluid between the first cavity region and the second cavity region; a first port and a second port each fluidly communicated with the first cavity region; and a third port and a fourth port each fluidly communicated with the second cavity region;
the housing has a substantially cylindrical configuration and the first port, the second port, the third port and the fourth port are disposed on the housing sequentially in an axially spaced manner from each other in an axial direction, preferably in a row; and
the second port and the third port are fluidly connected at least by the on-off valve.

22. The method according to claim 19, wherein the method further comprises:
controlling momentary peak pressure acting on the hollow fiber membranes within the second cavity region via a flow restriction connected fluidly in parallel with the on-off valve.

23. The method according to claim 17, wherein the flow restriction is configured as a variable flow restriction.

24. The method according to claim 18, wherein the method further comprises:
adjusting the substitution rate by further controlling the flow restriction.

25. The method according to claim 16, wherein the method further comprises:
adjusting a supplying speed of the medical fluid into the second cavity region at least based on the on-off operation of the on-off valve.

26. The method according to claim 17, wherein the method further comprises:
adjusting a supplying speed of the medical fluid into the second cavity region at least based on the on-off operation of the on-off valve.

27. The method according to claim 18, wherein the method further comprises:
adjusting a supplying speed of the medical fluid into the second cavity region at least based on the on-off operation of the on-off valve.

28. The method according to claim 19, wherein the method further comprises:
adjusting a supplying speed of the medical fluid into the second cavity region at least based on the on-off operation of the on-off valve.

29. The method according to claim 25, wherein
the supplying speed is reduced when the on-off valve is closed.

30. The method according to claim 25, wherein
the blood treatment system further comprises a fluid supply device, eg. a balancing chamber device (2) for supplying the medical fluid towards the dialyzer and the supplying speed is controlled by adjusting a speed of a flow pump (21) of the fluid supply device.

31. The method according to claim 19, wherein the method further comprises:
indirectly determining a first transmembrane pressure of the first cavity region based on a first pressure at the first port, a second pressure at the second port, a third pressure at the third port, a fourth pressure at the fourth port and a first set of additional parameters at least including a supplying flow rate of the medical fluid, the substitution rate and a blood flow rate, according to a first predetermined functional relationship; and/or
indirectly determining a second transmembrane pressure of the second cavity region based on a first pressure at the first port, a second pressure at the second port, a third pressure at the third port, a fourth pressure at the fourth port and a second set of additional parameters at least including a supplying flow rate of the medical fluid, the substitution rate and a blood flow rate, according to a second predetermined functional relationship.

32. The method according to claim 31, wherein
the first set of additional parameters further comprise at least one of an ultrafiltration factor and a hematocrit level; and/or
the second set of additional parameters further comprise at least one of an ultrafiltration factor and a hematocrit level.

33. The method according to claim 31, wherein
the first predetermined functional relationship is established by experiments and/or simulations; and/or
the second predetermined functional relationship is established by experiments and/or

34. The method according to claim 16, wherein the method further comprises:
monitoring the substitution rate by a flow sensor for detecting a flow rate of the medical fluid flowing from the second cavity region towards the first cavity region.

35. A blood treatment system, configured to be adapted to perform hemodialysis, hemofiltration and hemodiafiltration, comprising:
a memory with computer-readable program instructions stored thereon; and
a processor for executing the computer-readable program instructions so as to perform the method according to claim 16.
